(19) United States
(12) Patent Application Publication
(10) Pub. No.: US 2016/0011220 A1
(43) Pub. Date: Jan. 14, 2016

(54) Systems and Methods to Quantify Analytes in Keratinized Samples

(71) Applicant: PURDUE PHARMA L.P., Stamford, CT (US)

(72) Inventors: Paul Coplan, Plymouth Meeting, PA (US); Stephen Harris, Weston, CT (US); Craig Jay Landau, Branford, CT (US)

(21) Appl. No.: 14/377,632

(22) PCT Filed: Feb. 14, 2013
(86) PCT No.: PCT/US13/26199

§ 371 Date: Aug. 8, 2014

Related U.S. Application Data
(60) Provisional application No. 61/598,768, filed on Feb. 14, 2012.

(51) Int. Cl. G01N 33/94

(52) U.S. Cl. CPC G01N 33/9486

(57) ABSTRACT

Disclosed in certain embodiments is a method of analysis comprising quantifying the amount of a drug and/or drug metabolite in a keratinized sample of a subject that has been prescribed a dosing regimen of the drug to obtain a result; and comparing the result to a known standard value for the dosing regimen.

This application claims priority to U.S. Provisional Patent Application Ser. No. 61/598,768 filed Feb. 14, 2012, the disclosure of which is hereby incorporated by reference in its entirety for all purposes.

### FIELD OF THE INVENTION

The invention is directed to systems and methods to determine the presence of an analyte in a keratinized sample such as hair or nails.

### BACKGROUND OF THE INVENTION

One challenge of pharmacological therapy is to administer the active agent such that exposure to the agent is within the therapeutic window of the patient. Achieving exposure of the active agent above the therapeutic window can subject the patient to increased side-effects and toxicity. Conversely, if the therapeutic window of exposure is not reached, the patient does not receive the intended therapy at an effective dose which can lead to lack of efficacy morbidity and mortality.

Drugs that are susceptible to abuse (e.g., opioid analgesics) face these challenges as well as others given the illicit use of these agents by some subjects. A subject achieving drug exposure higher than intended may be an indication that the subject is abusing the substance and is taking doses more than intended for illicit, psychoactive or recreational use. Alternatively, a subject achieving drug exposure lower than intended may be an indication that the subject is diverting the substance from normal distribution channels and making the drug available to others for illicit use.

Another issue associated with therapy of drugs susceptible to abuse is the often unfair social stigma associated with these drugs. Even patients who have a legitimate clinical need for these agents are harassed and subjected to misplaced scrutiny by members of the public (including some health care professionals) who have a misconception or lack of understanding of the important role these agents have in the alleviation of suffering. Due to this stigma, many patients may not be completely forthcoming with their health care providers regarding their history of medication use. For example, they may understate the amount of medicine they are taking, regardless of the fact that the therapy is needed to manage their clinical state. Likewise, they may take less than the prescribed amount to avoid a misplaced negative perception about drug use, but are uneasy about admitting to a health care professional that they are not following a suggested treatment regimen.

Urine and blood tests may be administered in order ascertain the contemporaneous exposure of a subject to a drug. These tests are limited, however, as they only provide a snapshot of drug use, and do not provide a history showing drug exposure over an extended period of time. Instead, a health care professional often needs to rely on a personal interview with the patient in order to determine the extent of drug exposure by the subject over an extended period of time. Unfortunately, health care professionals cannot objectively verify this information.

There exists a need in the art for systems and methods to provide an analysis of recent drug exposure in subjects as compared to a known standard. There is a further need in the art for systems and methods to provide health care professionals with an objective test to determine long-term drug exposure of a subject as compared to a known standard that allows quantification of drug exposure relative to the intended dosage.

All references cited herein are incorporated by reference in their entireties for all purposes.

### OBJECTS AND SUMMARY

It is an object of certain embodiments of the present invention to provide systems and methods to verify the clinical effectiveness of drug therapy.

It is an object of certain embodiments of the present invention to provide systems and methods to verify the compliance of a subject with a prescribed dosing regimen.

It is an object of certain embodiments of the present invention to provide systems and methods to determine if drug therapy provides exposure above, within or below a therapeutic window.

It is an object of certain embodiments of the present invention to provide systems and methods to detect the illicit use of a drug.

It is an object of certain embodiments of the present invention to provide systems and methods to detect the diversion of a drug from standard distribution channels.

It is an object of certain embodiments of the present invention to provide systems and methods to determine the rate of drug metabolism of a subject.

It is an object of certain embodiments of the present invention to provide systems and methods to provide an objective measurement of drug exposure, especially in the context of medical disciplines such as pain management that often rely upon subjective measurements.

It is an object of certain embodiments of the present invention to provide systems and methods to quantify long term drug exposure in order to provide data for use in healthcare cost containment efforts.

The above objects of the present invention and others can be achieved by the present invention, which in certain embodiments is directed to a method of analysis comprising: quantifying the amount of a drug and/or drug metabolite in a keratinized sample from a subject that has been prescribed a dosing regimen of the drug, which quantifying produces a result; and comparing the result to a known standard value for the dosing regimen.

In certain embodiments, the present invention is directed to a method of analysis comprising: obtaining the result of a test quantifying the amount of a drug and/or drug metabolite in a keratinized sample of a subject that has been prescribed a dosing regimen of the drug; and comparing the result to a known standard value for the dosing regimen.

In certain embodiments, the present invention is directed to a method of screening for the illicit use of a drug in a subject comprising: quantifying the amount of the drug and/or drug metabolite in a keratinized sample of the subject and determining whether the result is higher than a known standard value for a dosing regimen of the drug previously prescribed for the subject; assessing the subject (e.g., by physical examination or interview) and determining that the subject is abusing the drug; and optionally reducing or discontinuing the prescribed dosing regimen.

In certain embodiments, the present invention is directed to a method of detecting the diversion of a drug in a subject comprising: obtaining a result of a test that quantifies the amount of the drug and/or drug metabolite in a keratinized sample of the subject and indicates that the result is lower than a known standard value for a dosing regimen of the drug previously prescribed for the subject; assessing the subject and determining that the subject is diverting the drug; and optionally reducing or discontinuing the prescribed dosing regimen.

In certain embodiments, the present invention is directed to a method of verifying the clinical effectiveness of drug therapy in a subject prescribed a drug comprising: obtaining a result of a test that quantifies the amount of the drug and/or drug metabolite in a keratinized sample of the subject and indicates that the result is higher than a known standard value for a dosing regimen of the drug previously prescribed for the subject; assessing the subject and determining that the subject is administering higher than the prescribed dose due to an increased clinical requirement; and optionally increasing the prescribed dosing regimen.

In certain embodiments, the present invention is directed to a method of verifying the clinical effectiveness of drug therapy in a subject prescribed a drug comprising: obtaining a result of a test that quantifies the amount of the drug and/or drug metabolite in a keratinized sample of the subject and indicates that the result is lower than a known standard value for a dosing regimen of the drug previously prescribed for the subject; assessing the subject and determining that the subject is administering lower than the prescribed dose due to a decreased clinical requirement; and optionally decreasing or discontinuing the prescribed dosing regimen.

In certain embodiments, the present invention is directed to a method of verifying compliance in a subject prescribed a drug comprising: obtaining a result of a test that quantifies the amount of the drug and/or drug metabolite in a keratinized sample of the subject and indicates that the result is higher than a known standard value for a dosing regimen of the drug previously prescribed for the subject; assessing the subject and determining that the subject is administering higher than the prescribed dose due to subject error; and optionally maintaining or decreasing the prescribed dosing regimen.

In certain embodiments, the present invention is directed to a method of verifying compliance in a subject prescribed a drug comprising: obtaining a result of a test that quantifies the amount of the drug and/or drug metabolite in a keratinized sample of the subject and indicates that the result is lower than a known standard value for a dosing regimen of the drug previously prescribed for the subject; assessing the subject and determining that the subject is administering lower than the prescribed dose due to subject error; and optionally maintaining or increasing the prescribed dosing regimen.

In certain embodiments, the present invention is directed to a method of determining the relative drug metabolism rate of a subject comprising: obtaining a result of a test that quantifies the amount of the drug and/or metabolite in a keratinized sample of the subject and indicates that the result is lower than a known standard value for a dosing regimen of the drug previously prescribed for the subject; assessing the subject and determining that the subject has an increased rate of metabolism of the drug when the results are lower than the known standard; and optionally increasing the prescribed dosing regimen.

In certain embodiments, the present invention is directed to a method of determining the relative drug metabolism rate of a subject comprising: obtaining a result of a test that quantifies the amount of the drug and/or a metabolite in a keratinized sample of the subject and indicates that the result is higher than a known standard value for a dosing regimen of the drug previously prescribed for the subject; assessing the subject and determining that the subject has a decreased rate of metabolism of the drug when the results are lower than the known standard; and optionally decreasing the prescribed dosing regimen.

In certain embodiments, one or more steps of the invention are performed by a module executable by a processing device.

In certain embodiments, the present invention is directed to a system comprising a module executable by a processing device for performing one or more steps of the methods disclosed herein.

The term “patient” means a subject, particularly a human, who has presented a clinical manifestation of a particular symptom or symptoms suggesting the need for treatment, who is treated preventatively or prophylactically for a condition, or who has been diagnosed with a condition to be treated. The term “subject” is inclusive of the definition of the term “patient” and does not exclude individuals who are entirely normal in all respects or with respect to a particular condition.

The term “keratinized sample” means a sample that contains keratin. Non limiting examples of such keratinized samples are hair, fingernails and toenails. A preferred sample is a hair sample.

The term “relative drug metabolism rate” means a comparison of a metabolism rate of an individual subject with the metabolism rate of a population of patients or subjects at a given time point or over a certain time period. An “increased metabolism rate” or “relatively fast metabolizer” means that the subject exhibits higher value(s) of the metabolite and lower value(s) of the drug in the sample in relation to the standard value obtained for the patient or subject population for the respective time point or time period. In one embodiment, the value obtained for the subject exhibiting an increased metabolism rate is at least 10% higher than the mean value obtained for the patient/subject population. A “decreased metabolism rate” or “relatively slow metabolizer” means that the subject exhibits higher value(s) of the drug and lower value(s) of the metabolite in the sample in relation to the standard value obtained for the patient or subject population for the respective time point or time period. In one embodiment the value obtained for the subject exhibiting a decreased metabolism rate is at least 10% lower than the mean value obtained for the patient/subject population.

The term “standard value” means a value based on a patient population value (optionally including one or two standard deviations) or on a personalized value of the subject (optionally including one or two standard deviations). The standard can be a specific value (e.g., based on a mean or median value) or a range (e.g., based on individual values or a standard deviation).

The term “drug is less active than the metabolite” means the drug has less effect on the biological target of interest than the compound created when the drug is metabolized by the body.

The term “drug is more active than the metabolite” means the drug has more effect on the biological target of interest than the compound created when the drug is metabolized by the body.

### BRIEF DESCRIPTION OF THE DRAWINGS

FIGS. 1A and 1B depict the hair levels of hydrocodone as compared to the total daily dose of hydrocodone for a population of subjects from visits one and two as disclosed in Example 1.

FIGS. 2A and 2B depict the hair levels of oxycodone as compared to the total daily dose of oxycodone for a population of subjects from visits one and two as disclosed in Example 1.

FIGS. 3A and 3B depict the hair levels of morphine as compared to the total daily dose of morphine for a population of subjects from visits one and two as disclosed in Example 1.

FIGS. 4A and 4B depict the hair levels of hydrocodone as compared to the total daily dose of hydrocodone for a sub-population of subjects from visits one and two as disclosed in Example 1.

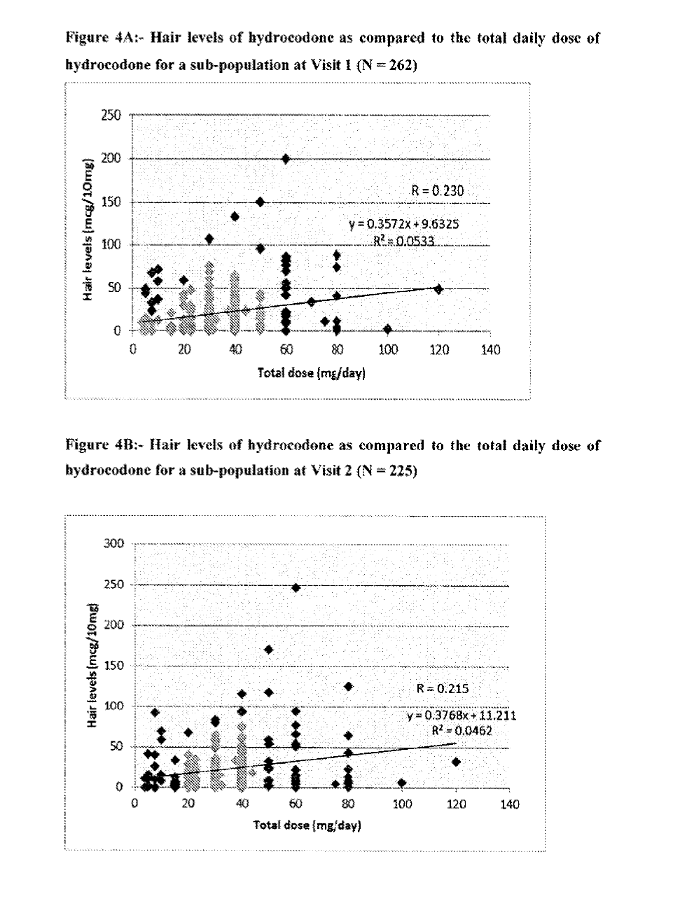

FIGS. 5A and 5B depict the hair levels of oxycodone as compared to the total daily dose of oxycodone for a sub-population of subjects from visits one and two as disclosed in Example 1.

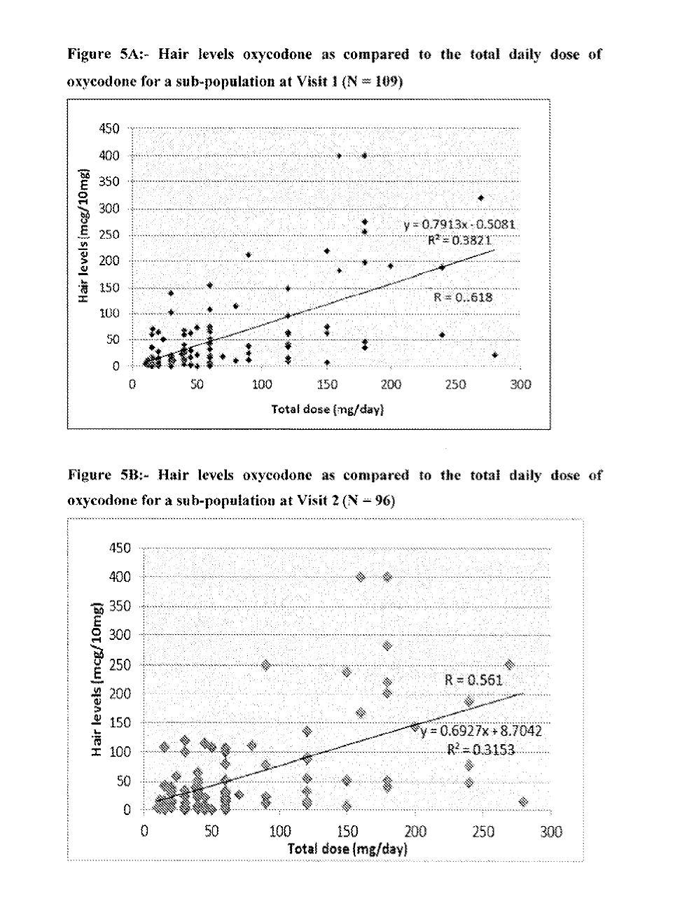

FIGS. 6A and 6B depict the hair levels of morphine as compared to the total daily dose of morphine for a sub-population of subjects from visits one and two as disclosed in Example 1.

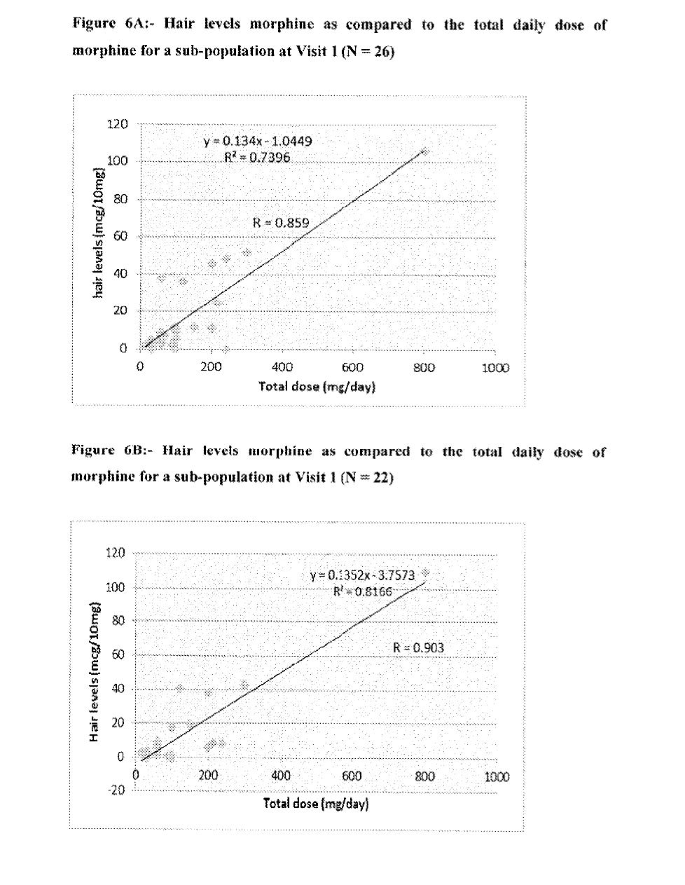

FIGS. 7A and 7B compares the hydrocodone hair levels from visits one and two of individuals on stable dosages of the drug as disclosed in Example 1.

FIGS. 8A and 8B compares the oxycodone hair levels from visits one and two of individuals on stable dosages of the drug as disclosed in Example 1.

FIGS. 9A and 9B compares the morphine hair levels from visits one and two of individuals on stable dosages of the drug as disclosed in Example 1.

### DETAILED DESCRIPTION

Typical prior art analyses of keratinized samples (e.g., hair, fingernails or toenails) from a subject for the presence of a pharmacological agent are binary measurements, i.e., they established either the presence or absence of the agent. These tests were often used to detect drugs of abuse in connection with employment requirements or drug rehabilitation matters, and they indicated whether the subject had or had not taken the particular agent. Such tests, however, are inadequate to elucidate the extent or timing of exposure to the agent, such as the amount of the dose and the length of time over which administration may have occurred.

By virtue of the present invention, the extent of exposure to the agent can be quantifiably measured in order to provide health practitioners with an objective tool to manage the overall healthcare of the subject, to determine compliance with a prescribed regimen of a controlled drug, and to make an objective assessment as to the actual abuse or diversion of prescribed drugs. The methods and systems of the present invention can be used to determine exposure not only to drugs susceptible to abuse, but also to non-controlled drugs that are utilized for various chronic and acute conditions.

By virtue of the present invention, there is disclosed a method of analysis comprising: quantifying the amount of a drug (or drug metabolite) in a keratinized sample of a subject that has been prescribed a dosing regimen of the drug, which quantifying produces a result; and comparing the result to a known standard value for the dosing regimen. The known standard value can be a single point or a range (e.g., based on the standard deviation).

Once administered, the drug may be metabolized to a less active metabolite or to a more active metabolite. The drug, the metabolite or both may be present in the keratinized sample. If a subject is a “relatively slow metabolizer” of the drug, then relatively more of the drug and less of the metabolite will be present in the sample. If a subject is a “relatively fast metabolizer” of the drug, then relatively less of the drug and more of the metabolite will be present in the sample.

The analysis of the keratinized sample can be used to quantify the drug, or the metabolite, or both the drug and the metabolite.

Any of the methods of the present invention can be performed by, e.g., health care professionals, laboratory centers, medical payors (e.g., health maintenance organizations, pharmacy benefit managers) or other individuals or entities that may have an interest in quantifying the exposure of a subject to a particular active agent. A medical payor may perform the test, e.g., to determine compliance with a dosing regimen in order to provide better outcomes and reduced costs.

In performing any of the methods of the invention, a keratinized sample (e.g., hair or nails) is collected and analyzed to determine the quantity of an active agent therein. The active agent can either be the drug that has been prescribed to the patient according to a particular dosing regimen, or a metabolite of said drug. The quantitative result is then compared to a known standard value. Depending on whether the value is above, below, equal to the standard value, or within a standard value range, provides an objective tool to assess the subject. The known standard value of a particular dosing regimen can be based on a patient population value (optionally including one or two standard deviations), or on a personalized value of the subject. The known standard can be a specific value (e.g., based on a mean or median value) or a range (e.g., based on individual values or a standard deviation).

A patient population standard value may be determined by administering a specific amount of a drug according to a dosing regimen to a patient population (e.g., 2, 10, 50, 100, or 1,000 or more subjects). A keratinized sample is then obtained from the patient at a predetermined time point, or at periodic time points, and analyzed for the presence of the drug or drug metabolite. In certain embodiments, the periodic measurements are performed until the drug or drug metabolite is at steady state in the keratinized sample. Calculations can then be made in order to determine the mean values and obtain a standard value or range wherein a certain percentage (e.g., at least 70%, at least 80%, at least 85%, at least 90%, at least 95% or at least 99%) of the population will fall within when administered the drug according to the dosing regimen. When a keratinized sample of an individual subject is analyzed for a drug that has been prescribed according to a particular regimen, a determination can then be made as to whether the subject's test result is above, below or within the standard value, and an overall assessment (e.g., by physical examination or interview) of the subject can then be made as to the cause. In certain embodiments a determination is made as to the difference in the amount of the drug and/or the metabolite between the known standard value and the sample. For example, it can be determined that the sample contains an amount of drug and/or metabolite that is greater than 10% above or below the standard value or less than 10% above or below the standard value. A determination can then be made on taking further action (e.g., increasing or decreasing the drug dose) if the sample contains an amount of the drug and/or drug metabolite that is above or below the standard value by a particular threshold, e.g., plus or minus 1%, 2%, 5%, 8%, 10%, 15% or 20%. A determination can also be made on taking further action (e.g., increasing or decreasing the dose) if the sample contains an amount of the drug or drug metabolite that is above or below the standard value for two or more testing intervals.

A personalized standard value for a particular subject can be determined by administering a specific amount or range of a drug according to a dosing regimen to the subject. A keratinized sample is then collected from the subject at a predetermined time point, or at periodic time points, and analyzed to determine quantity of the drug and/or drug metabolite. In certain embodiments, the periodic measurements are performed until the drug and/or drug metabolite is at steady state in the individual subject in the keratinized sample. This provides a real time baseline for the individual patient such that when keratinized samples of the individual subject are subsequently analyzed for the drug and/or drug metabolite, a determination can be made as to whether the results are above, below or within the personalized value, and an overall assessment (e.g., by physical examination or personal interview) of the subject can then be made as to the cause of any departure from the personalized value.

The standard value can also be determined at the initial onset of therapy prior to the patient initiating the dosing regimen to establish a baseline. The baseline may also be used to verify whether the patient has provided accurate information as to past use. If a patient or subject is opioid naive, the initial value would be zero. If a patient or subject has been on chronic opioids, a baseline can be determined at the onset of the testing period or the initiation of new therapy.

If analysis of the keratinized sample produces a result that is higher than the known standard (i.e., the individual or population standard), then an assessment of the subject can be made that the subject is self-administering, or a care-giver is administering to the subject, a dose of the drug that is higher than the prescribed regimen. A care-giver includes but is not limited to a healthcare provider (e.g., a nurse), social worker, friend, relative, volunteer, etc.

The administration of a higher dose than prescribed can be due to abuse (e.g., illicit and/or recreational use). In this situation, the health care provider can respond by reducing or discontinuing the dosing regimen.

In other situations, the administration of a higher dose than prescribed can be due to a manifestation of an increased clinical need by the subject (e.g., increased pain), or a perceived increased clinical need by a care-giver caring for the subject. In this situation, the health care provider can respond by increasing the dosing regimen.

In other situations, the administration of a higher dose than prescribed can be due to dosing errors by the subject or a care-giver. In this situation, the health care provider can respond by decreasing the regimen. A patient or care-giver education plan can also be implemented.

If the drug is the more active species (compared to the metabolite), then certain conclusions may be reached based on analysis of the keratinized structure. If the analysis of the keratinized sample produces a result showing the presence of a higher amount of the drug than the known standard, then an assessment of the subject can be made that the subject is a relatively slow metabolizer of the drug. In this situation, the health care provider can respond by reducing the drug dosage or discontinuing the dosing regimen. Alternatively, if the analysis of the keratinized sample produces a result showing the presence of a lower amount of the drug than the known standard, then an assessment of the subject can be made that the subject is a relatively fast metabolizer of the drug. In this situation, the health care provider can respond by increasing the drug dosage. If the analysis of the keratinized sample produces a result showing the presence of a higher amount of the metabolite than the known standard, then an assessment of the subject can be made that the subject is a relatively fast metabolizer of the drug. In this situation, the health care provider can respond by maintaining or increasing the drug dosage. Alternatively, if the analysis of the keratinized sample produces a result showing the presence of a lower amount of the metabolite than the known standard, then an assessment of the subject can be made that the subject is a relatively slow metabolizer of the drug. In this situation, the health care provider can respond by reducing the drug dosage or discontinuing the dosing regimen.

If the metabolite is the more active species (compared to the drug), then certain conclusions may be reached based on analysis of the keratinized structure. If the analysis of the keratinized sample produces a result showing the presence of a higher amount of the drug than the known standard, then an assessment of the subject can be made that the subject is a relatively slow metabolizer of the drug. In this situation, the health care provider can respond by increasing the drug dose. Alternatively, if the analysis of the keratinized sample produces a result showing the presence of a lower amount of the drug than the known standard, then an assessment of the subject can be made that the subject is a relatively fast metabolizer of the drug. In this situation, the health care provider can respond by reducing the drug dose or discontinuing the dosing regimen. If the analysis of the keratinized sample produces a result showing the presence of a higher amount of the metabolite than the known standard, then an assessment of the subject can be made that the subject is a relatively fast metabolizer of the drug. In this situation, the health care provider can respond by reducing the drug dose or discontinuing the dosing regimen. Alternatively, if the analysis of the keratinized sample produces a result showing the presence of a lower amount of the metabolite than the known standard, then an assessment of the subject can be made that the subject is a relatively slow metabolizer of the drug. In this situation, the health care provider can respond by increasing the drug dose.

In certain embodiments, a ratio of drug to metabolite can be established in order to make a determination whether a subject is a slow or fast metabolizer.

If the analysis of the keratinized sample produces a result that is lower than the known standard, then an assessment of the subject can be made to determine whether the subject is self-administering, or being administered by a care-giver, a lower amount (or no amount) of the drug than the prescribed regimen.

The self-administration or administration by a care-giver, of a lower dose than prescribed can reflect diversion of the drug from the intended subject. In this situation, the health care provider can respond by reducing or discontinuing the dosing regimen.

In other situations, the self-administration, or administration by a care-giver, of a lower dose than prescribed can be due to a manifestation or perception of a decreased clinical need of the patient (e.g., decreased pain). In this situation, the health care provider can respond by decreasing or discontinuing the dosing regimen.

In other situations, the administration of a lower dose than prescribed can be due to error on the part of the subject or care-giver. In this situation, the health care provider can respond by maintaining or increasing the regimen. A patient or care-giver education plan can also be implemented.

If the analysis of the keratinized sample produces a result that is lower than the known standard, then an assessment of the subject can be made to determine whether the subject is a relatively high metabolizer of the drug as compared to the known standard value. In this situation, the health care provider can respond by increasing the dosing regimen.

When the quantification of the keratinized sample is at, or within, the known standard or known standard range, the dosing regimen can be maintained.

### Quantification Tests

The present invention can utilize any known method for quantification of an analyte in a keratinized sample, e.g., as described in Suzuki et al., Forensic Sci. International, 24:9-16, 1984; A. W. Holmes, Textile Research Journal, 706-712, August 1964; Annette M. Baumgartner, et al., Journal of Nuclear Medicine, 20:748-752, 1979; D. Valente, et al., Clinical Chemistry, Vol. 27, No. 11, 1981; A. M. Baumgartner, et al., Journal of Forensic Sciences, p. 576-81, July 1981; Smith et al., Journal of Forensic Sciences, Vol. 26, No. 3, July 1981, pp. 582-586; W. A. Baumgartner et al., J. Nucl Med 23:790-892, 1982; Ishiyama, et al., Journal of Forensic Sciences, Vol. 28, No. 2, April 1983, pp. 380-385; K. Puschel, et al., Forensic Science International, 21 (1983) 181-186; 0. Suzuki, et al., Journal of Forensic Sciences, Vol. 29, No. 2, April 1984, pp. 611-617; N. J. Haley et al., Clin. Chem. 31/10, 1598-1600 (1985); Sramek et al., A. M. J. Psychiatry 142:8, August 1985; Baumgartner, et al., Clinical Nuclear Medicine, vol. 10, September 1985; Gill, et al., Nature, Vol. 318, p. 577 (1985); Smith et al., J. Forensic Sci. 1986, 31(4), 1269-73; M. Margio, et al., “Determination of Morphine and Other Opioids in the Hair of Heroin Addicts by HPLC and MS/MS”, International Conference, University of Verona, Jun. 25-26, 1986; M. Marigo, et al., Journal of Analytical Toxicology, Vol. 10, July/August 1986; M. Michalodinitrakis, Med. Sci. Law (1987), Vol. 27, No. 1; Pelli, et al., Biomedical and Environmental Mass Spectrometry, Vol. 14, 63-68 (1987); and Higuchi et al., Nature, Vol. 332, p. 543 (1988).

Other methods that can be utilized in the quantification step of the present invention include, e.g., those described in U.S. Pat. No. 5,324,642; U.S. Pat. No. 5,466,579; U.S. Pat. No. 6,022,693; U.S. Pat. No. 6,350,582; U.S. Pat. No. 6,582,924; U.S. Pat. No. 6,949,344; U.S. Pat. No. 8,084,215; U.S. Patent Application Publication No. 2009/0269791; and U.S. Patent Application Publication No. 2011/0104714.

In certain embodiments, the quantification step of the present invention can include subjecting the keratinized sample to hot methanol solutions and by overnight incubation of hair in an alkaline or acid medium. These methods can also be performed in combination with physical and/or chemical pulverization steps.

After release from the keratinized sample, the analyte can be assayed with a suitable instrument or procedure, e.g., a radioimmunoassay, a gas chromatograph, an HPLC and/or a mass spectrometer.

In certain embodiments, the quantification step of the present invention can include the step of dissolving of hair samples by exposure, e.g., to sodium hydroxide and heat, followed by analysis for the presence of the analyte by, e.g., a radioimmunoassay.

In certain embodiments, the quantification step of the present invention can include detection of the analyte by gas chromatography and chemical ionization mass spectrometry after treatment with, e.g., a sodium hydroxide solution to which has been added N-methylbenzylamine.

In certain embodiments, the quantification step of the present invention can include dissolving the sample in a buffer solution containing gelatin, sodium chloride, Tris (tris(hydroxymethyl)aminomethane) and EDTA (ethylenediaminetetraacetic acid), followed by conducting an assay, e.g., a radioimmunoassay.

In certain embodiments, the quantification step of the present invention can include quantitative determination of the analyte in a keratinized sample with heat-acid hydrolysis, pre-column dansyl derivatization, straight phase liquid chromatography and fluorescence detection.

In certain embodiments, the quantification step of the present invention can include subjecting the sample to an organic solvent, such as diethylether and an acid such as hydrochloric acid, followed by dissolution of the dried extract in a suitable solvent such as methanol.

In certain embodiments, the quantification step of the present invention can include contacting the keratinized sample with a mixture containing a low-redox potential compound such as dithiothreitol (DTT) or dithioerythritol (DTE) and an enzyme suitable for the dissolution of the keratinized sample. The resultant solution can then be analyzed to quantify the analyte. The enzyme can be, e.g., peptidase, endopeptidase, protease, papain, chymopapain, or proteinase K. Optionally, cupric sulfate or sodium arsenite can be added to the solution to deactivate interfering excess dithiothreitol or dithioerythritol. An assay of the analyte can then be performed with, e.g., an immunoassay.

In other embodiments, the quantification step of the present invention can include contacting the keratinized sample with a reducing agent without any contact with a proteolytic agent, in order to reduce disulfide bonds present in the keratinized sample without substantially cleaving peptide bonds. The steps may include reducing the sample, deactivating the process and an optional purification step by, e.g., filtration or centrifugation. The reducing agent can be, e.g., DTT, DTE, thioglycolate, cysteine, sulfites, bisulfites, sulfides, bisulfides or TCEP (tris(2-carboxyethyl)phosphine), or salt forms of any of the foregoing.

In certain embodiments, the correlation coefficient (r) (which measures the direction of the linear relationship between total daily dose and sample levels) between a first analyte measurement or a sample and a second analyte measurement of a sample at a later time point (e.g., as disclosed in Example 1) is greater than about 0.2; greater than about 0.25 greater than about 0.3 greater than about 0.35; greater than about 0.4; greater than about 0.45; greater than about 0.5; greater than about 0.55; greater than about 0.6; greater than about 0.65; greater than about 0.7 greater than about 0.75; greater than about 0.8; greater than about 0.85; or greater than about 0.9.

In certain embodiments, the coefficient of determination (r squared) (which provides the proportion of the variance between total daily dose and sample levels) between a first analyte measurement of a sample and a second analyte measurement of a sample at a later time point (e.g., as disclosed in Example 1) is greater than about 0.04; greater than about 0.06 greater than about 0.08; greater than about 0.1; greater than about 0.2; greater than about 0.3; greater than about 0.4; greater than about 0.5; greater than about 0.6; greater than about 0.7; or greater than about 0.8.

In certain embodiments, the p-value (which demonstrates if the correlation coefficient is statistically significant) between a first analyte measurement of a sample and a second analyte measurement of a sample at a later time point (e.g., as disclosed in Example 1) is less than about 0.2, less than about 0.01; less than about 0.001; or less than about 0.0001.

The first measurement and the second measurement used for the statistical calculations can be any two analyte measurements collected during a time interval. The measurements can be sequential or have intervening measurement within the time interval. The first measurement used in the statistical measurement can be an initial analyte measurement of a subject or any subsequent measurement.

### Active Agents and Disease States

In certain embodiments, the analyte that is detected according to the present invention may be selected from the group consisting of ACE inhibitors, adenohypophyseal hormones, adrenergic neuron blocking agents, adrenocortical steroids, inhibitors of the biosynthesis of adrenocortical steroids, alpha-adrenergic agonists, alpha-adrenergic antagonists, selective alpha-two-adrenergic agonists, analgesics, anti-pyretics, anti-inflammatory agents, androgens, local and general anesthetics, anti-addictive agents, anti-androgens, anti-arrhythmic agents, anti-asthmatic agents, anti-cholinergic agents, anti-cholinesterase agents, anti-coagulants, anti-diabetic agents, anti-diarrheal agents, anti-diuretic, anti-emetic and pro-kinetic agents, anti-epileptic agents, anti-estrogens, anti-fungal agents, anti-hypertensive agents, anti-microbial agents, anti-migraine agents, anti-muscarinic agents, anti-neoplastic agents, anti-parasitic agents, anti-parkinson's agents, anti-platelet agents, anti-progestins, anti-schizophrenia agents, anti-thyroid agents, anti-tussives, anti-viral agents, atypical anti-depressants, azaspirodecanediones, barbiturates, benzodiazepines, benzothiadiazides, beta-adrenergic agonists, beta-adrenergic antagonists, selective beta-one-adrenergic antagonists, selective beta-two-adrenergic agonists, bile salts, agents affecting volume and composition of body fluids, butyrophenones, agents affecting calcification, calcium channel blockers, cardiovascular drugs, catecholamines and sympathomimetic drugs, cholinergic agonists, cholinesterase reactivators, contraceptive agents, dermatological agents, diphenylbutylpiperidines, diuretics, ergot alkaloids, estrogens, ganglionic blocking agents, ganglionic stimulating agents, hydantoins, agents for control of gastric acidity and treatment of peptic ulcers, hematopoietic agents, histamines, histamine antagonists, hormones, 5-hydroxytryptamine antagonists, drugs for the treatment of hyperlipoproteinemia, hypnotics, sedatives, immunosupressive agents, laxatives, methylxanthines, moncamine oxidase inhibitors, neuromuscular blocking agents, organic nitrates, opioid agonists, opioid antagonists, pancreatic enzymes, phenothiazines, progestins, prostaglandins, agents for the treatment of psychiatric disorders, retinoids, sodium channel blockers, agents for spasticity and acute muscle spasms, succinimides, testosterones, thioxanthines, thrombolytic agents, thyroid agents, tricyclic antidepressants, inhibitors of tubular transport of organic compounds, drugs affecting uterine motility, vasodilators, vitamins, metabolites thereof and mixtures thereof.

In certain embodiments, the analyte that is detected according to the present invention is an opioid agonist or a metabolite thereof. In such embodiments, the opioid agonist is selected from the group consisting of alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, proheptazine, promedol, properidine, propiram, propoxyphene, sufentanil, tilidine, tramadol, pharmaceutically acceptable salts thereof, and mixtures thereof. In certain embodiments, the opioid agonist is selected from the group consisting of codeine, fentanyl, hydromorphone, hydrocodone, oxycodone, dihydrocodeine, dihydromorphine, morphine, tramadol, oxymorphone, pharmaceutically acceptable salts thereof, metabolites thereof, and mixtures thereof. In certain embodiments, the analyte is oxycodone, a metabolite thereof, or a pharmaceutically acceptable salt thereof.

The opioid metabolite that can be detected may be, e.g., a metabolite formed by O-dealkylation, N-dealkylation, ketoreduction, deacetylation, glucuronidation or sulfatation of the opioid drug administered to the subject.

When testing for morphine, an exemplary assay includes testing for morphine, codeine and/or 6-acetylmorphine (used to differentiate between heroin and morphine as 6-acetyl morphine is a metabolite of heroin, not morphine, so its presence indicates heroin).

When testing for hydrocodone, an exemplary assay includes testing for hydrocodone, codeine and/or hydromorphone.

When testing for oxycodone, an exemplary assay includes testing for oxycodone and oxymorphone.

In certain embodiments, the analyte that is detected according to the present invention is an opioid antagonist or a metabolite thereof. In such embodiments, the opioid antagonist is selected from the group consisting of amiphenazole, naltrexone, methylnaltrexone, naloxone, nalbuphine, nalorphine, nalorphine dinicotinate, nalmefene, nadide, levallorphan, cyclozocine, pharmaceutically acceptable salts thereof, metabolites thereof; and mixtures thereof.

In other embodiments, the analyte that is detected according to the present invention is a non-opioid analgesic or a metabolite thereof. In such embodiments, the non-opioid analgesic is a non-steroidal anti-inflammatory agent selected from the group consisting of aspirin, celecoxib, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam, isoxicam, pharmaceutically acceptable salts thereof, and mixtures thereof.

In certain embodiments, the analyte that is detected according to the present invention is a benzodiazepine, barbiturate or amphetamine, an antagonist thereof, a metabolite thereof, or a combination thereof.

Benzodiazepines can be selected from alprazolam, bromazepam, chlordiazepoxide, clorazepate, diazepam, estazolam, flurazepam, halazepam, ketazolam, lorazepam, nitrazepam, oxazepam, prazepam, quazepam, temazepam, triazolam, and pharmaceutically acceptable salts, hydrates, and solvates thereof, and mixtures thereof. Benzodiazepine antagonists include, but are not limited to, flumazenil and pharmaceutically acceptable salts, hydrates, and solvates thereof.

Barbiturates include, but are not limited to, amobarbital, aprobarbotal, butabarbital, butalbital, methohexital, mephobarbital, metharbital, pentobarbital, phenobarbital, secobarbital and pharmaceutically acceptable salts, hydrates, and solvates thereof, and mixtures thereof. Barbiturate antagonists include, but are not limited to, amphetamines and pharmaceutically acceptable salts, hydrates, and solvates thereof.

In certain embodiments, the analyte that is detected according to the present invention is a stimulant or metabolite thereof. Stimulants include agents such as amphetamine, dextroamphetamine resin complex, dextroamphetamine, methamphetamine, methylphenidate, pharmaceutically acceptable salts, hydrates, and solvates thereof, and mixtures thereof. In certain embodiments, the analyte that is detected according to the present invention is a stimulant antagonist or metabolite thereof. Stimulant antagonists include, but are not limited to, benzodiazepines, and pharmaceutically acceptable salts, hydrates, and solvates thereof.

### Systems

Systems of the present invention can include a machine with a computer system within which a set of instructions for causing the machine to perform any one or more of the methodologies discussed herein may be executed. In some embodiments, the machine may be connected (e.g., networked) to other machines in a LAN, an intranet, an extranet, or the Internet. The machine may operate in the capacity of a server machine in client-server network environment. The machine may be a personal computer (PC), a set-top box (STB), a server, a network router, switch or bridge, or any machine capable of executing a set of instructions (sequential or otherwise) that specify actions to be taken by that machine. The machine can also be a portable device such as a hand held computer, tablet or smartphone with wireless network connectivity. Further, the term “machine” shall also be taken to include any collection of machines that individually or jointly execute a set (or multiple sets) of instructions to perform any one or more of the methodologies discussed herein.

The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### Example 1

### Study Design and Plan for Evaluating the Use of Hair Testing for Managing Opioid Analgesics and their Safe Use in Patients Treated for Chronic Pain in Community-Based Practices

An ongoing study has been implemented on adult female and male patients treated for chronic pain that have been prescribed oxycodone, hydrocodone or morphine. The study includes 2 study visits approximately 60 days apart. The second visit allowable range is between 45 and 75 days after the first visit. Patients submit hair and urine samples at both study visits

A. Sample Collection

Two urine samples are collected with one sent to a central diagnostics laboratory and the other tested according to the standard practice of each clinical site.

Hair samples are obtained and sent to a central diagnostics laboratory and tested for cocaine, codeine, heroine (identified by 6-acetylmorphine), morphine, oxycodone (with its metabolite of oxymorphone), hydrocodone (with its metabolite of hydromorphone), hydromorphone, phencyclidine, ecstasy, amphetamine and marijuana.

The enrolled subjects are current, past or new users of prescribed opioid analgesic regimens in either (i) the past 30 days, (ii) the past 31 to 120 days or were not previously prescribed an opioid regimen. All opioid analgesic regimens are self-reported.

B. Inclusion Criteria

Provide written informed consent.

Males and females 18 years and older.

Prescribed oxycodone, hydrocodone or morphine.

Willing and able to participate in study.

C. Exclusion Criteria

Head hair shorter than one-half inch in length.

Patients prescribed Embeda® (morphine sulfate and naltrexone hydrochloride extended release capsules).

Investigator believes the patient to be unsuitable.

D. Relationship Between Prescribed Opioid Dose and Hair Levels (Excluding PRN Dose)

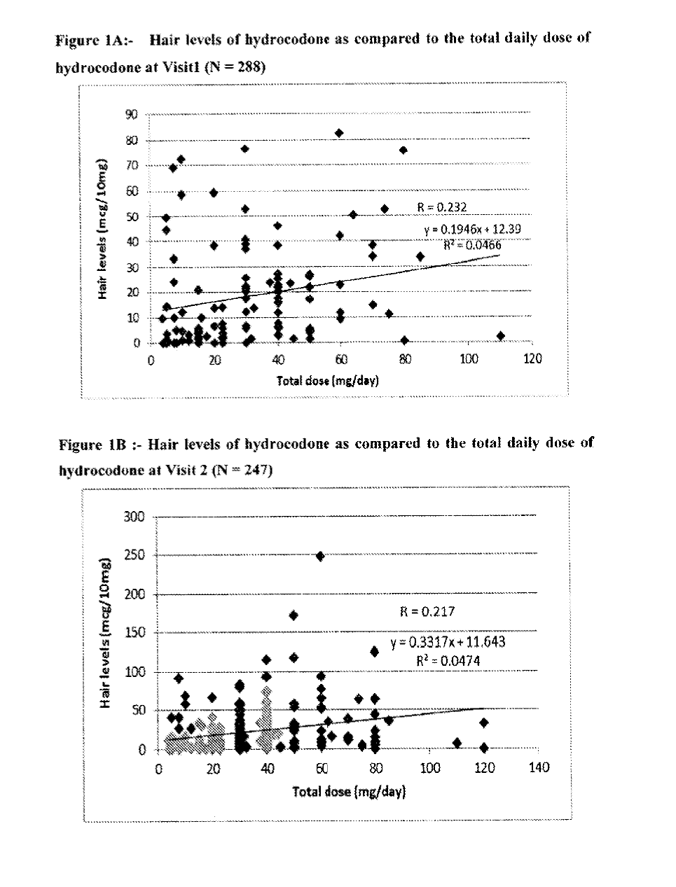

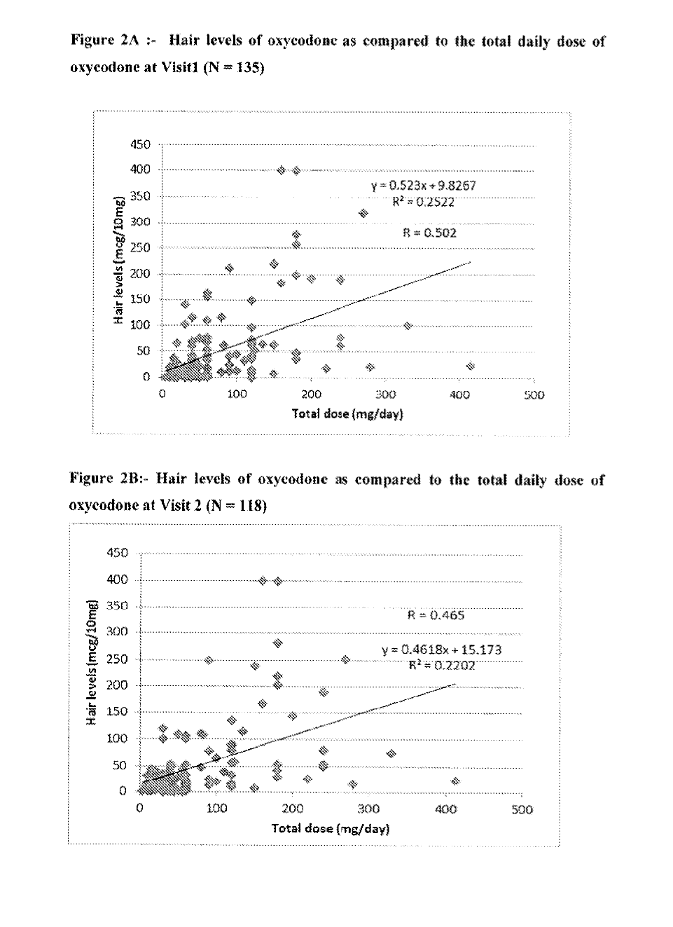

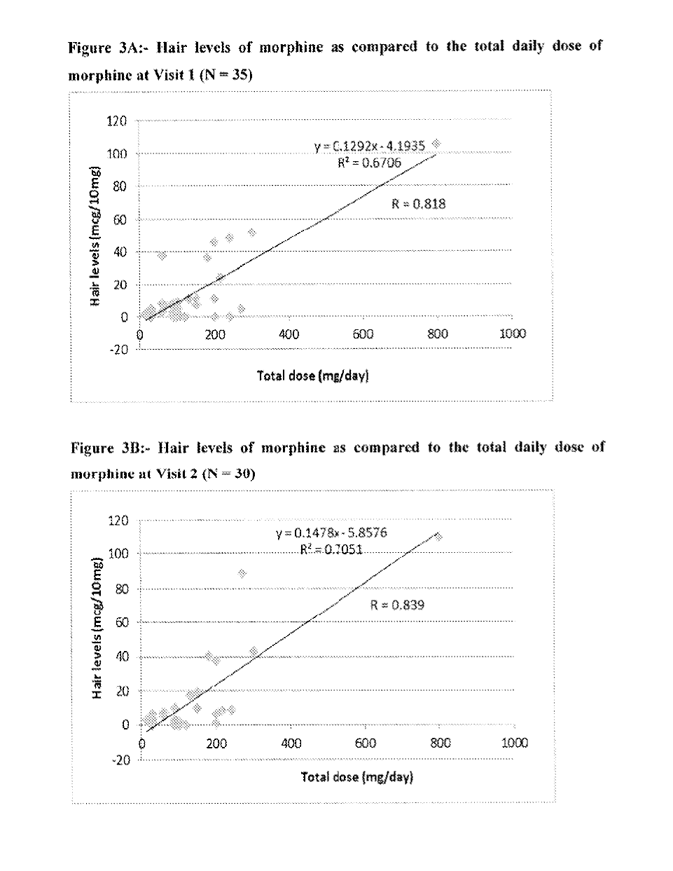

A comparison of the hair levels of subjects prescribed either hydrocodone, oxycodone or morphine to daily dosing was recorded at each of visits 1 and 2. The total dose (mg/day) was graphically plotted against the amount of drug/metabolite found in the hair sample (hair levels) (μ/10 mg) for hydrocodone in FIG. 1A; for oxycodone in FIG. 2A; and for morphine in FIG. 3A. Data from each graphical depiction (n, r-squared, r and p value) are tabulated in Table 1B. Also tabulated in Table 1A is a sub-population of tested subjects which excluded patients where the start date of the regimen was within the 90 day window of the enrollment date. This sub-population of subjects is graphically plotted for hydrocodone in FIG. 1B; for oxycodone in FIG. 2B; and for morphine in FIG. 3B.

TABLE 1A

| | | | | | |
|---|---|---|---|---|---|
| | | | R- | r (correlation | |
| Opioid | Visit | N | Squared | coefficient) | P value |

All interim sample
(n = 411)

| | | | | | |
|---|---|---|---|---|---|
| Hydrocodone | 1 | 288 | 0.046 | 0.232 | <.0001 |
| Hydrocodone | 2 | 247 | 0.047 | 0.217 | .0006 |
| Oxycodone | 1 | 135 | 0.252 | 0.502 | <.0001 |
| Oxycodone | 2 | 118 | 0.220 | 0.465 | <.0001 |
| Morphine | 1 | 35 | 0.670 | 0.818 | <.0001 |
| Morphine | 2 | 30 | 0.705 | 0.839 | <.0001 |

Excluding patient-dose whose prescription started less than 90 days before
enrollment (n = 361)

| | | | | | |
|---|---|---|---|---|---|
| Hydrocodone | 1 | 262 | 0.053 | 0.230 | 0.0006 |
| Hydrocodone | 2 | 225 | 0.046 | 0.215 | 0.0011 |
| Oxycodone | 1 | 109 | 0.382 | 0.618 | <.0001 |
| Oxycodone | 2 | 96 | 0.315 | 0.561 | <.0001 |
| Morphine | 1 | 26 | 0.739 | 0.859 | 0.0467 |
| Morphine | 2 | 22 | 0.816 | 0.903 | 0.038 |

As shown in Table 1A and the accompanying Figures, the correlation coefficient (r), which measures the direction of the linear relationship between total daily dose and hair levels shows a positive correlation between the two variables.

The correlation coefficient for the tested agents was not changed significantly when the group of subjects starting a dosing regimen with 90 days of enrollment was excluded. This demonstrates that the linear relationship between the variables does not overly depend on when the prescribed regimen was initiated.

The coefficient of determination (r squared) provides the proportion of the variance (fluctuation) of one variable that is predictable from the other variable, and measures the strength of the linear relationship between the two variables (total daily dose v. hair levels). As it depends on the r value, the coefficient of determination (r squared) for the tested agents was similar between the group of total subjects and the group which excluded subjects starting a dosing regimen with 90 days of enrollment.

The coefficient of determination for hydrocodone shows that approximately 10% of the total variation in the hair levels can be explained by the linear relationship between the total daily dose vs. hair levels with the remaining variation being unexplained.

The coefficient of determination for oxycodone shows that approximately 55% of the total variation in the hair levels can be explained by the linear relationship between the total daily dose v. hair levels with the remaining variation being unexplained.

The p-value depicted in Table 1A and the accompanying graphs demonstrate that the correlation coefficient is statistically significant.

E. Assessment of the Stability of Hair Levels Among Individuals on Stable Doses of Opioids (Excluding PRN Dose)

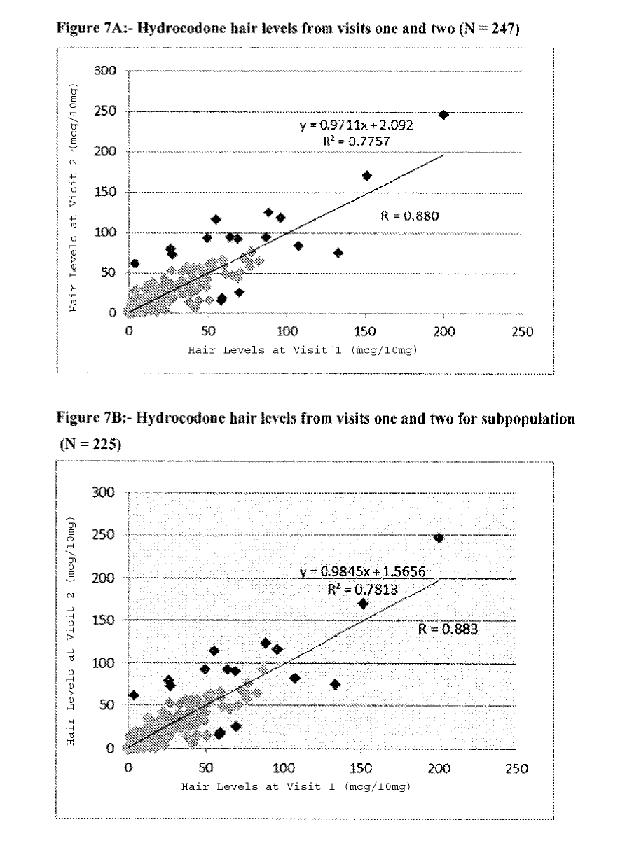

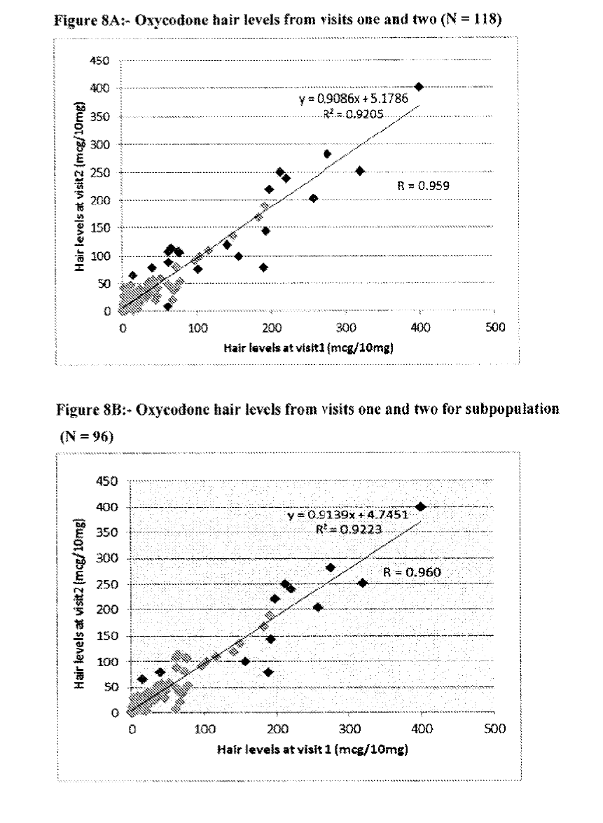

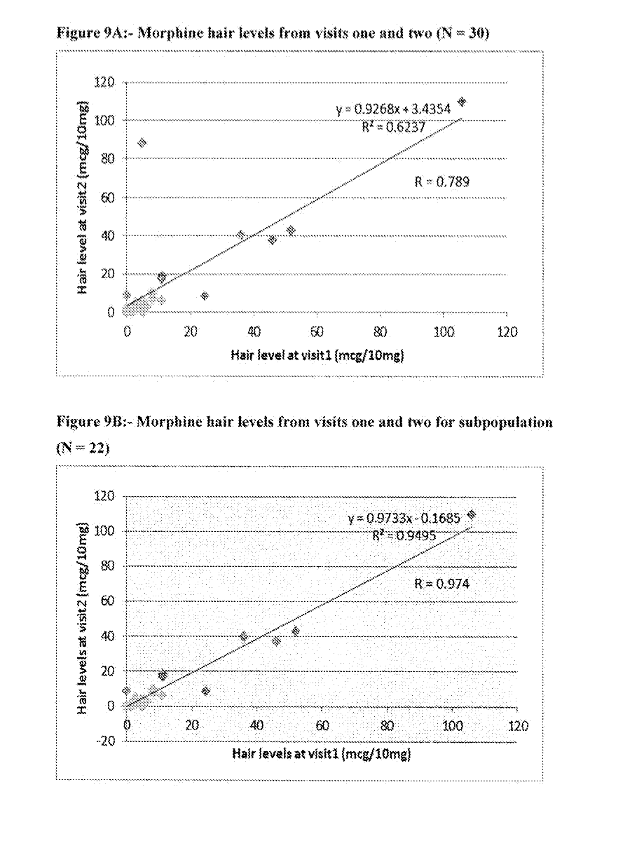

A comparison of the hair levels of subjects prescribed hydrocodone, oxycodone or morphine at visit 1 were compared to the hair levels of the respective drugs at visit 2. The hair level at visit 1 was graphically plotted against the hair level at visit 2 for hydrocodone in FIGS. 7A and 7B; for oxycodone in FIGS. 8A and 8B; and for morphine in FIGS. 9A and 9B. Data from each graphical depiction (n, r-squared, r and p value) are tabulated in Table 1B. Also shown in Table 1B and the graphs is data from a sub-population of tested subjects, which excluded patients where the start date of the regimen was within the 90 day window of the enrollment date.

TABLE 1B

| | | | | | |
|---|---|---|---|---|---|
| | | | R- | R (correlation | |
| Opioid | Visit 1 | Visit 2 | Squared | coefficient) | P value |

All interim sample (n = 411)

| | | | | | |
|---|---|---|---|---|---|
| Hydrocodone | 288 | 247 | 0.775 | 0.880 | <.0001 |
| Oxycodone | 135 | 118 | 0.920 | 0.959 | <.0001 |
| Morphine | 35 | 30 | 0.623 | 0.789 | <.0001 |

Excluding patients whose prescription started less than 90 days before
enrollment (n = 361)

| | | | | | |
|---|---|---|---|---|---|
| Hydrocodone | 262 | 225 | 0.781 | 0.883 | <.0001 |
| Oxycodone | 109 | 96 | 0.922 | 0.960 | <.0001 |
| Morphine | 26 | 22 | 0.949 | 0.974 | <.0001 |

As shown in Table 1B and FIGS. 7-9, there is a high correlation between the hair levels obtained between visits 1 and 2 when a subject is on a stable dosing regimen of opioid.

### Example 2

### Prophetic

1. A patient is prescribed 5 mg oxycodone hydrochloride every 6 hours for severe pain;
2. On weekly follow-up visits, the physician assesses the overall health of the patient and takes a hair sample;
3. The hair sample is quantified for the amount of oxycodone or oxycodone metabolite contained therein per specific unit of hair sample;
4. Step three is repeated until at least two successive quantifications are not substantially different (e.g., with 5% or 10% or 15% of each other), thus establishing a personalized standard value (e.g., a single value or a range) for the prescribed dosing regimen;
5. On subsequent visits, a hair sample is quantified and compared to the personalized standard value;
6. Depending on the comparison to the personalized standard value in conjunction with an assessment of the patient, the physician or other professional has a measure of whether the patient's ingestion of opioid has changed (increased or decreased). If hair levels are increased relative to the personal standard, the prescriber can investigate whether the patient may be abusing the analgesic or has interference in metabolism of the analgesic; if hair levels are decreased, the prescriber can investigate whether the patient may by diverting the analgesic or is not adhering to the therapeutic regimen of the analgesic. Consequently the prescriber may choose to monitor the patient, as well as decrease, discontinue, maintain, or increase the opioid analgesic as appropriate.

### Example 3

### Prophetic

1. A patient is prescribed 5 mg oxycodone hydrochloride every 6 hours for severe pain;
2. On weekly follow-up visits, the physician assesses the overall health of the patient and takes a hair sample;
3. The hair sample is quantified for the amount of oxycodone or oxycodone metabolite contained therein per specific unit of hair sample;
4. The quantification of oxycodone in the hair sample(s) is compared to a patient population standard for the prescribed dosing regimen at particular time points;
5. Depending on the comparison to the patient population standard value in conjunction with an assessment of the patient, the physician or other professional has a measure of whether the patient's ingestion of opioid has changed (increased or decreased). If hair levels are increased relative to the patient population standard, the prescriber can investigate whether the patient may be abusing the analgesic or has interference in metabolism of the analgesic; if hair levels are decreased, the prescriber can investigate whether the patient may by diverting the analgesic or is not adhering to the therapeutic regimen of the analgesic. Consequently, the prescriber may choose to monitor the patient, as well as decrease, discontinue, maintain, or increase the opioid analgesic as appropriate.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

1. A method of analysis comprising:
(i) quantifying the amount of a drug or a drug metabolite in a keratinized sample of a subject that has been prescribed a dosing regimen of the drug to obtain a result; and
(ii) comparing the result to a known standard value for the dosing regimen.

2. The method of claim 1, wherein the known standard value is a patient population value.

3. The method of claim 1, wherein the known standard value is a personalized value of the subject.

4. The method of claim 1, wherein the result is higher than the known standard value.

5. The method of claim 1, wherein the result is lower than the known standard value.

6. The method of claim 1, wherein the result is at or within the known standard value.

7. The method of claim 4, further comprising (iii) factoring the result into an assessment of the subject and determining that the subject is administering a higher amount of the drug than the prescribed dosing regimen.

8. The method of claim 7, wherein the assessment indicates that the administering of the higher amount is due to abuse.

9. The method of claim 7, wherein the assessment indicates that the administering of the higher amount is due to a manifestation of an increased clinical need of the patient.

10. The method of claim 7, wherein the assessment indicates that the administering of the higher amount is due to dosing error on the part of the subject or the caregiver.

11. The method of claim 8, wherein the dosing regimen is reduced or discontinued.

12. The method of claim 9, wherein the dosing regimen is increased.

13. The method of claim 10, wherein the dosing regimen is maintained or decreased.

14. The method of claim 4, further comprising (iii) factoring the result into an assessment of the subject and determining that the subject is a relatively slow metabolizer of the drug as compared to the known standard value.

15. The method of claim 14, wherein the dosing regimen is reduced or discontinued.

16. The method of claim 5, further comprising (iii) factoring the result into an assessment of the subject and determining that the subject is administering a lower amount of the drug than the prescribed regimen.

17. The method of claim 16, wherein the assessment indicates that the administering of the lower amount is due to diversion of the drug.

18. The method of claim 16, wherein the assessment indicates that the administering of the lower amount is due to a manifestation of a decreased clinical need of the subject.

19-31. (canceled)

32. A method of detecting the illicit use of a drug in a subject comprising:
(i) obtaining a result of a test that quantifies the amount of drug or drug metabolite in a keratinized sample of the subject and indicates that the result is higher than a known standard value for a dosing regimen for that drug previously prescribed for the subject;
(ii) assessing the subject and determining that the subject is abusing the drug; and optionally
(iii) reducing or discontinuing the prescribed dosing regimen.

33. (canceled)

34. A method of verifying the clinical effectiveness of drug therapy in a subject prescribed a drug comprising:
(i) obtaining a result of a test that quantifies the amount of drug or drug metabolite in a keratinized sample of the subject and indicates that the result is higher than a known standard value for a dosing regimen previously prescribed for the subject;
(ii) assessing the subject and determining that the subject is administering higher than the prescribed dose due to an increased clinical requirement; and optionally
(iii) increasing the prescribed dosing regimen.

35-64. (canceled)
